(19) United States
(12) Patent Application Publication
(10) Pub. No.: US 2023/0044425 A1
(43) Pub. Date: Feb. 9, 2023

(54) PROCESS FOR METHANOL PRODUCTION FROM INERT-RICH SYNTHESIS GAS

(71) Applicant: SABIC Global Technologies B.V., Bergen op Zoom (NL)

(72) Inventors: Abdulaziz Saad ALARIFI, Riyadh (SA); Awais AHMED, Riyadh (SA); Mubarik Ali BASHIR, Riyadh (SA)

(21) Appl. No.: 17/789,751

(22) PCT Filed: Dec. 30, 2020
(86) PCT No.: PCT/IB2020/062546

§ 371 Date: Jun. 28, 2022

(30) Foreign Application Priority Data
Jan. 2, 2020 (EP) 20150099.8

(51) Int. Cl. C07C 29/154

(52) U.S. Cl. CPC C07C 29/154

(57) ABSTRACT

Systems and methods for producing methanol are disclosed. A synthesis gas stream is split to form a first synthesis gas stream and a second synthesis gas stream. The first synthesis gas stream is fed into a primary methanol synthesis unit. The second synthesis gas stream is fed into a secondary methanol synthesis unit for producing methanol. The first effluent stream from the primary methanol synthesis unit and/or the second effluent stream from the secondary methanol synthesis unit are further separated to produce methanol product stream and one or more recycle streams comprising hydrogen, carbon monoxide, carbon dioxide and an inert gas. The one or more recycle streams are recycled to the primary methanol synthesis unit.

### CROSS REFERENCE TO RELATED APPLICATIONS

This application claims the benefit of priority of European Patent Application No. 20150099.8, filed Jan. 2, 2020, which is hereby incorporated by reference in its entirety.

### FIELD OF INVENTION

The present invention generally relates to methanol production processes. More specifically, the present invention relates to systems and methods for producing methanol using inert-rich synthesis gas.

### BACKGROUND OF THE INVENTION

Methanol is a highly versatile chemical used in many areas of the chemical industry. For example, methanol is commonly used as feedstock in the manufacture of various chemicals, including plastics, paints, plywood, biodiesels, and textiles. Furthermore, methanol can also be used as a denaturant, a solvent, and an antifreeze reagent. Further yet, many specialized vehicles have been developed to consume methanol as an alternative fuel either in combination with gasoline or alone.

Currently, the majority of methanol in the chemical industry is produced from synthesis gas (syngas). As shown in system 10 in FIG. 1, in the methanol synthesis process, carbon monoxide and/or carbon dioxide react with hydrogen in methanol synthesis reactor 101 to produce methanol via the following reactions: CO+2H₂⇔CH₃OH (1); CO₂+3H₂⇔CH₃OH+H₂O (2); and CO+H₂O⇔CO₂+H₂ (3). The unreacted carbon monoxide, carbon dioxide, and hydrogen in first effluent stream 14 of methanol synthesis reactor 101 is then recycled back to methanol synthesis reactor 101. However, synthesis gas feed stream 30 (also referred to as makeup-gas) often contains inert gases such as nitrogen and/or methane gas. Thus, after separation of methanol from first effluent stream 14 of methanol synthesis reactor 101 in first separation unit 109, recycle stream 23 is further divided into first recycle stream 24 and purge portion 25. Purge portion 25 is then separated to produce permeate gas stream 27. Both first recycle stream 24 and permeate gas stream 27, which contain higher inerts than synthesis gas feed stream 30, are recycled back to methanol synthesis reactor 101, leading to limited driving force of methanol synthesis reactions by shifting the equilibrium line toward less rate of methanol synthesis reactions. Therefore, the methanol production efficiency for the conventional system and method is generally low.

Overall, while the systems and methods for producing methanol using synthesis gas exist, the need for improvements in this field persists in light of at least the aforementioned drawback of the conventional systems and methods.

### BRIEF SUMMARY OF THE INVENTION

A solution to at least some of the above-mentioned problems associated with the systems and methods for producing methanol has been discovered. The solution resides in a method of producing methanol that includes reacting most of a synthesis gas feed stream in a primary methanol synthesis unit to produce methanol and reacting a portion of a synthesis gas feed stream in a secondary methanol synthesis unit to produce additional methanol and lower the load on the primary methanol synthesis unit. This can be beneficial as it increases the total volume of the catalyst in the methanol synthesis loop, resulting in increased methanol production efficiency. Additionally, the recycle gas from both the primary methanol synthesis unit and the secondary methanol synthesis unit can be recycled to the primary methanol synthesis unit to prevent the influent stream of secondary methanol synthesis unit from having a high inert gas content, resulting in improved methanol production efficiency. Furthermore, operating the secondary methanol synthesis unit without an in-flowing recycle stream can reduce the catalyst volume used in the secondary methanol synthesis unit, resulting in reduced operating cost and/or capital expenditure. Therefore, the method of the present invention provides a technical solution to at least some of the problems associated with the conventional systems and methods for producing methanol mentioned above.

Embodiments of the invention include a method of producing methanol. The method includes providing a synthesis gas feed stream comprising carbon oxides (including carbon monoxide, and carbon dioxide), hydrogen, and inert gases and dividing the synthesis gas feed stream to form a first synthesis gas stream and a second synthesis gas stream. The method comprises in a primary methanol synthesis unit, subjecting the first synthesis gas stream to reaction conditions sufficient to produce a first effluent stream comprising methanol, unreacted carbon oxides, unreacted hydrogen, and the inert gases from the synthesis gas feed stream. The method comprises in a secondary methanol synthesis unit, subjecting the second synthesis gas stream to reaction conditions sufficient to produce a second effluent stream comprising methanol, unreacted carbon oxides, unreacted hydrogen, and the inert gases from the synthesis gas feed stream. The method comprises separating the methanol from unreacted syngas and the inert gas from the first effluent stream and/or from the second effluent stream to produce a first recycle stream comprising primarily unreacted carbon oxides, unreacted hydrogen, and the inert gases, collectively, and a permeate gas stream comprising primarily unreacted carbon oxides and unreacted hydrogen, collectively. The method comprises flowing the first recycle stream and the permeate gas stream to the primary methanol synthesis unit and/or secondary methanol synthesis unit.

Embodiments of the invention include a method of producing methanol. The method includes providing a synthesis gas feed stream comprising carbon oxides (including carbon monoxide, and carbon dioxide), hydrogen, and inert gases. The synthesis gas feed stream comprises 5 to 25 mol. % inert gas. The method includes dividing the synthesis gas feed stream to form a first synthesis gas stream and a second synthesis gas stream. The method comprises, in a primary methanol synthesis unit, subjecting the first synthesis gas stream to reaction conditions sufficient to produce a first effluent stream comprising methanol, unreacted carbon oxides, unreacted hydrogen, and some of the inert gas from the synthesis gas feed stream. The method comprises, in a secondary methanol synthesis unit, subjecting the second synthesis gas stream to reaction conditions sufficient to produce a second effluent stream comprising methanol, unreacted carbon oxides, unreacted hydrogen, and the inert gases from the synthesis gas feed stream. The method comprises separating the methanol from unreacted syngas and at least some of the inert gas from the first effluent stream and/or from the second effluent stream to produce a first recycle stream comprising primarily unreacted carbon oxides, unreacted hydrogen, and the inert gases, collectively, and a permeate gas stream comprising primarily unreacted carbon oxides and unreacted hydrogen, collectively. The first unreacted syngas stream comprises 5 to 25 mol. % inert gas. The method comprises flowing the first recycle stream and the permeate gas stream to the primary methanol synthesis unit and/or secondary methanol synthesis unit.

Embodiments of the invention include a method of producing methanol. The method includes providing a synthesis gas feed stream comprising carbon oxides (including carbon monoxide, and carbon dioxide), hydrogen, and an inert gas. The synthesis gas feed stream comprises 5 to 25 mol. % inert gas. The method includes dividing the synthesis gas feed stream to form a first synthesis gas stream and a second synthesis gas stream. The method comprises, in a primary methanol synthesis unit, subjecting the first synthesis gas stream to reaction conditions sufficient to produce a first effluent stream comprising methanol, unreacted carbon oxides, unreacted hydrogen, and some of the inert gas from the synthesis gas feed stream. The method comprises, in a secondary methanol synthesis unit, subjecting the second synthesis gas stream to reaction conditions sufficient to produce a second effluent stream comprising methanol, unreacted carbon oxides, unreacted hydrogen, and some of the inert gas from the synthesis gas in the synthesis gas feed stream. The method further comprises separating the first effluent stream in a first separation unit to produce a first unreacted syngas stream and a first crude methanol stream. The method further comprises separating the second effluent stream in a second separation unit to produce a second recycle stream and a second crude methanol stream. The method further still comprises dividing the first unreacted syngas stream to form the first recycle stream and a purge gas separation unit (PGSU) first feed gas stream. The method further comprises separating the purge gas separation unit (PGSU) first feed gas stream and at least a portion of second unreacted syngas stream in an inert separation unit to form (i) a permeate gas stream comprising primarily carbon oxides and hydrogen, collectively and (ii) a residue gas stream comprising primarily the inert gas. The method comprises flowing the first recycle stream and permeate gas stream to the primary methanol synthesis unit.

The following includes definitions of various terms and phrases used throughout this specification.

The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

The terms “wt. %”, “vol. %” or “mol. %” refer to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, include any measurable decrease or complete inhibition to achieve a desired result.

The term “effective,” as that term is used in the specification and/or claims, means adequate to accomplish a desired, expected, or intended result.

The term “stoichiometric number of hydrogen to carbon monoxide” or “S_{N}” as that term is used in the specification and/or claims, refers to a ratio of [(H₂—CO₂)/(CO+CO₂)], where (H₂—CO₂) is the molar concentration difference between hydrogen and carbon dioxide in a mixture or a stream, and (CO+CO₂) is the molar concentration sum between carbon monoxide and carbon dioxide in a mixture or a stream.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

For a more complete understanding, reference is now made to the following descriptions taken in conjunction with the accompanying drawings, in which:

FIG. 1 shows schematic diagram of a conventional system for producing methanol;

FIGS. 2A and 2B show schematic diagrams of systems for producing methanol, according to embodiments of the invention. FIG. 2A shows a system for producing methanol, in which an effluent from a primary methanol synthesis unit is processed in a same separation unit as an effluent from a secondary methanol synthesis unit; FIG. 2B shows a system for producing methanol, in which an effluent from a primary methanol synthesis unit flows to a different separation unit than an effluent from a secondary methanol synthesis unit; and

FIG. 3 shows a schematic flowchart for a method of producing methanol, according to embodiments of the invention.

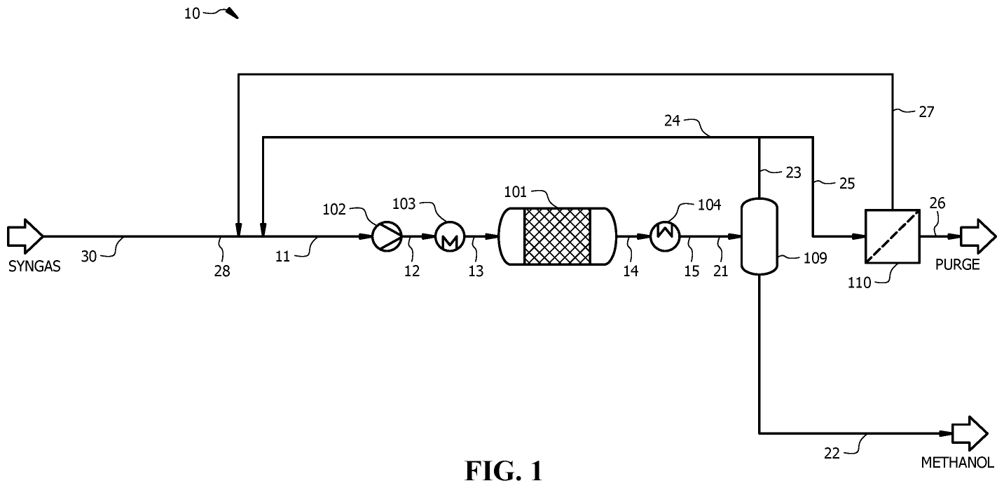

### DETAILED DESCRIPTION OF THE INVENTION

A method has been discovered for producing methanol using syngas in a system that includes a primary methanol synthesis unit for producing methanol and a secondary methanol synthesis unit to produce additional methanol. A synthesis gas stream is divided to form a first synthesis gas stream and a second synthesis gas stream, which are fed into the primary methanol synthesis unit and the secondary methanol synthesis unit, respectively. The effluent stream from the primary methanol synthesis unit and/or the effluent stream from the secondary methanol synthesis unit are separated to form one or more recycle streams, which are flowed to the primary methanol synthesis unit. Notably, the additional secondary methanol synthesis unit is capable of increasing the total volume of the catalyst in the system, thereby increasing the methanol production efficiency. Additionally, a first unreacted syngas stream, which contains a higher percentage of inert gas than the feed synthesis gas stream, can be fed into the primary methanol synthesis unit or both the primary methanol synthesis unit and the secondary methanol synthesis unit. Thus, the concentration of inert gas fed into the secondary methanol synthesis unit can be lower than the inert gas concentration of a feed stream of a conventional methanol synthesis reactor, which includes a combined stream of a synthesis make up stream and a recycle gas stream, thereby further increasing the methanol production efficiency. These and other non-limiting aspects of the present invention are discussed in further detail in the following sections.

### A. System for Producing Methanol

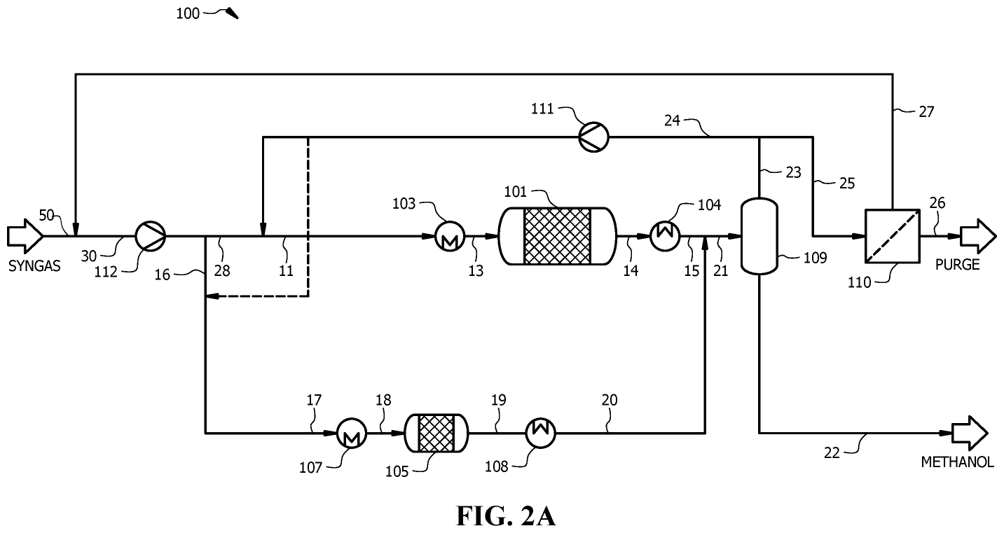

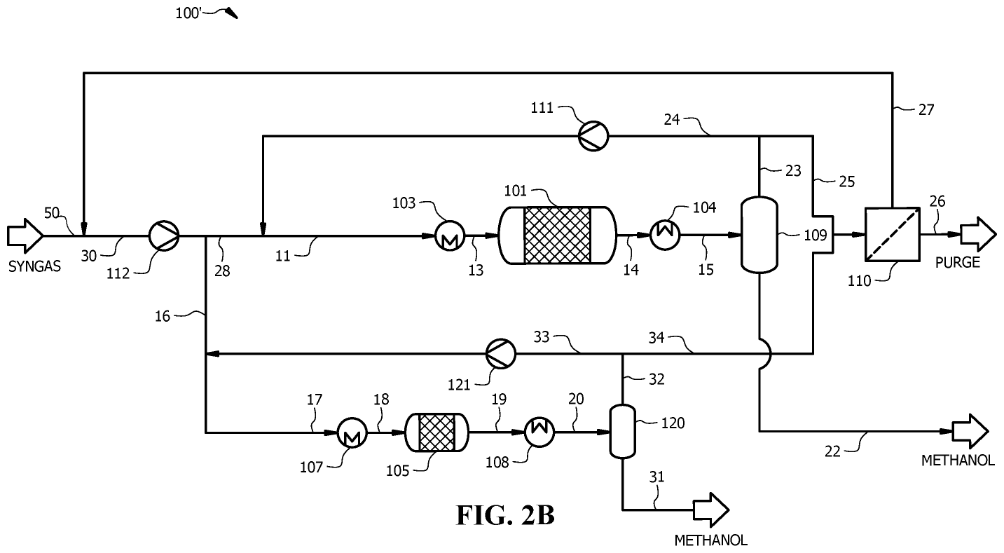

In embodiments of the invention, a system for producing methanol includes a primary methanol synthesis unit, a secondary methanol synthesis unit, and one or more separation units. With reference to FIGS. 2A and 2B, schematic diagrams are shown of systems 100 and 100′, respectively, for producing methanol.

According to embodiments of the invention, system 100 comprises primary methanol synthesis unit 101 adapted to receive first feed stream 11 comprising carbon oxides (including carbon monoxide and/or carbon dioxide), hydrogen, and inert gases, and to react the carbon oxides and hydrogen to produce methanol. In embodiments of the invention, primary methanol synthesis unit 101 comprises one or more fixed bed reactors in parallel and/or in series and/or a combination of in parallel and in series. Primary methanol synthesis unit 101 may comprise a catalyst capable of catalyzing synthesis of methanol by a reaction of hydrogen with carbon oxides. The catalyst may comprise Cu, Zn, Al₂O₃, or combinations thereof.

According to embodiments of the invention, system 100 comprises first preheater 103 located upstream of primary methanol synthesis unit 101. In embodiments of the invention, first preheater 103 may be configured to heat first feed stream 11 to a temperature of 140 to 240° C. to form preheated feed stream 13. An outlet of first preheater 103 feeds to primary methanol synthesis unit 101.

According to embodiments of the invention, an outlet of primary methanol synthesis unit 101 feed first cooler 104 such that first effluent stream 14 flows from primary methanol synthesis unit 101 to first cooler 104. First effluent stream 14 may comprise methanol, the inert gas, unreacted hydrogen, unreacted carbon oxides, or combinations thereof. First cooler 104 may be configured to cool first effluent stream 14 to form first cooled effluent stream 15 at a temperature of 20 to 120° C. and all ranges and values there between including ranges of 20 to 30° C., 30 to 40° C., 40 to 50° C., 50 to 60° C., 60 to 70° C., 70 to 80° C., 80 to 90° C., 90 to 100° C., 100 to 110° C., and 110 to 120° C.

According to embodiments of the invention, system 100 comprises secondary methanol synthesis unit 105 configured to receive second synthesis gas stream 16 and react hydrogen and carbon oxides of second synthesis gas stream 16 in the presence of a catalyst to produce methanol. The catalyst in secondary methanol synthesis unit 105 can be the same or substantially the same as the catalyst in primary methanol synthesis unit 101. In embodiments of the invention, secondary methanol synthesis unit 105 comprises one or more adiabatic or isothermal reactors in series. In embodiments of the invention, the total reactor volume of secondary methanol synthesis unit 105 is 5 to 25% of the total reactor volume of primary methanol synthesis unit 101.

System 100 may further comprise second preheater 107. Second preheater 107 may be configured to heat second synthesis gas stream 16 to form second preheated synthesis gas stream 18 at a temperature of 140 to 240° C. An outlet of second preheater 107 may be in fluid communication with an inlet of secondary methanol synthesis unit 105 such that second preheated synthesis gas stream 18 flows from second preheater 107 to secondary methanol synthesis unit 105.

According to embodiments of the invention, an outlet of secondary methanol synthesis unit 105 feeds to second cooler 108 such that second effluent stream 19 flows from secondary methanol synthesis unit 105 to second cooler 108. In embodiments of the invention, second effluent stream 19 from secondary methanol synthesis unit 105 includes methanol, the inert gas, unreacted hydrogen, unreacted carbon oxides, or combinations thereof. Second cooler 108 may be configured to cool second effluent stream 19 to form second cooled effluent stream 20 at a temperature of 20 to 120° C. and all ranges and values there between including ranges of 20 to 30° C., 30 to 40° C., 40 to 50° C., 50 to 60° C., 60 to 70° C., 70 to 80° C., 80 to 90° C., 90 to 100° C., 100 to 110° C., and 110 to 120° C.

In embodiments of the invention, second cooled effluent stream 20 and first cooled effluent stream 15 are combined to form combined effluent stream 21. According to embodiments of the invention, system 100 includes first separation unit 109 configured to separate combined effluent stream 21 to form (i) first crude methanol stream 22 comprising primarily methanol, (ii) first unreacted syngas stream 23 comprising primarily unreacted hydrogen, unreacted carbon oxides (including carbon monoxide and carbon dioxide), and the inert gas. In embodiments of the invention, first unreacted syngas stream 23 includes 5 to 25 mol. % of the inert gas and all ranges and values there between including ranges of 5 to 10 mol. %, 10 to 15 mol. %, 15 to 20 mol. %, and 20 to 25 mol. %.

In embodiments of the invention, first unreacted syngas stream 23 is separated to form first recycle stream 24 and purge gas separation unit (PGSU) first feed gas stream 25. According to embodiments of the invention, system 100 includes hydrogen membrane unit 110 configured to separate most of the hydrogen and some of the unreacted carbon oxides from purge gas separation unit (PGSU) first feed gas stream 25 to produce residue gas stream 26 comprising primarily the inert gas and permeate gas stream 27 comprising primarily carbon oxides and hydrogen, collectively. A flow rate of purge gas separation unit (PGSU) first feed gas stream 25 to first unreacted syngas stream 23 is in a range of 0 to 20% and all ranges and values there between including ranges of 0 to 2%, 2 to 4%, 4 to 6%, 6 to 8%, 8 to 10%, 10 to 12%, 12 to 14%, 14 to 16%, 16 to 28%, and 18 to 20%. Flow rate of permeate gas stream 27 may be determined by an amount of hydrogen required in primary methanol synthesis unit 101 and/or secondary methanol synthesis unit 105. Hydrogen membrane unit 110 may be replaced by a pressure swing adsorption unit, or any other gas separation unit.

According to embodiments of the invention, first recycle stream 24 is combined with first synthesis gas stream 28 to form first feed stream 11 for primary methanol synthesis unit 101. As an alternative to or in addition to being combined with first synthesis gas stream, at least a portion of first recycle stream 24 may be combined with second synthesis gas stream 16 to form second feed stream 17. In embodiments of the invention, each of first synthesis gas stream 28 and second synthesis gas stream 16 is a portion of synthesis gas feed stream 30. In embodiments of the invention, system 100 comprises first recycle compressor 111 configured to compress first recycle stream 24 prior to combining with first synthesis gas stream 28. According to embodiments of the invention, permeate gas stream 27 may be combined with crude synthesis gas stream 50 to form synthesis gas seed stream 30. Synthesis gas feed stream 30 may be compressed by feed compressor 112 before it is split into first synthesis gas stream 28 and second synthesis gas stream 16.

According to embodiments of the invention, as shown in FIG. 2B, system 100′ includes all the equipment and units in system 100 except that second cooled effluent stream 20 of system 100′ does not combine with first cooled effluent stream 15. In embodiments of the invention, system 100′ includes second separation unit 120. An outlet of second cooler 108 may be in fluid communication with an inlet of second separation unit 120 such that second cooled effluent stream 20 flows from second cooler 108 to second separation unit 120. Second separation unit 120 may be configured to separate second cooled effluent stream 20 into (i) second crude methanol stream 31 comprising primarily methanol and (ii) second unreacted syngas stream 32 comprising primarily unreacted hydrogen, unreacted carbon oxides, and the inert gas, collectively. In embodiments of the invention, second unreacted syngas stream 32 is divided into second recycle stream 33 and purge gas separation unit (PGSU) second feed gas stream 34. Second recycle stream 33 may be combined with second synthesis gas stream 16 to form second feed stream 17. In embodiments of the invention, recycle stream 33 is compressed by second recycle compressor 121 before it is combined with second synthesis gas stream 16. Purge gas separation unit (PGSU) second feed gas stream 34 may be flowed to inert separation unit 110. A flow rate ratio of second recycle stream 33 and purge gas separation unit (PGSU) second feed gas stream 34 may be determined based on an amount of hydrogen required in secondary methanol synthesis.

### B. Method of Producing Methanol

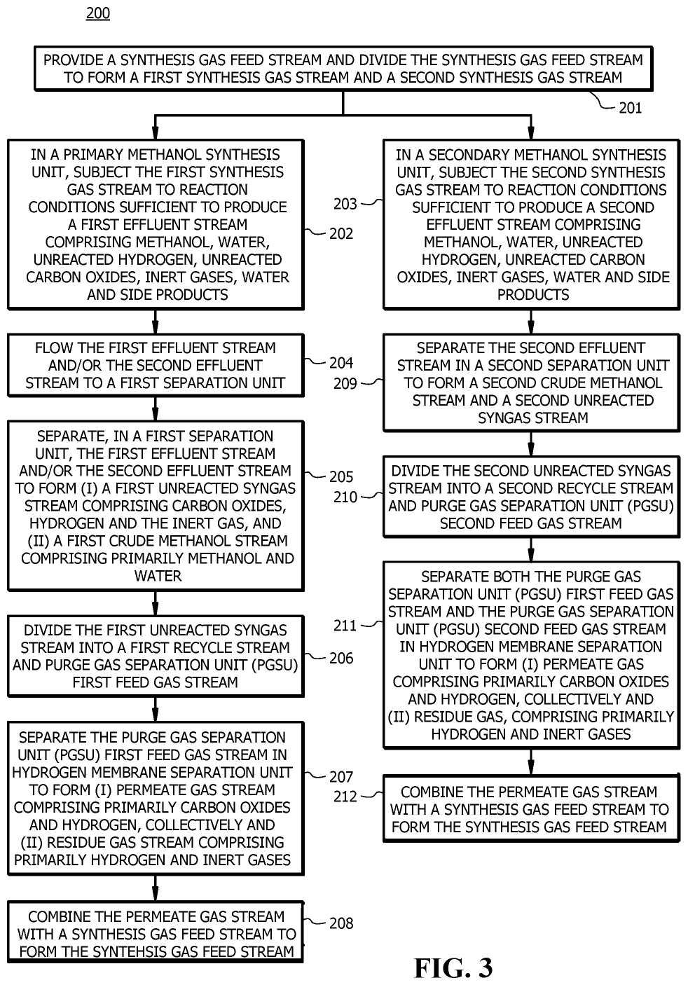

Methods of producing methanol using synthesis gas have been discovered. Embodiments of the methods are capable of improving methanol production efficiency compared to conventional methods. As shown in FIG. 3, embodiments of the invention include method 200 for producing methanol. Method 200 may be implemented by system 100 and/or system 100′, as shown in FIG. 2A and FIG. 2B, respectively.

According to embodiments of the invention, as shown in block 201, method 200 includes providing synthesis gas feed stream 30 and dividing synthesis gas feed stream 30 to form first synthesis gas stream 28 and second synthesis gas stream 16. In embodiments of the invention, synthesis gas feed stream 30 comprises hydrogen, carbon monoxide, carbon dioxide, and the inert gas. The inert gas may include nitrogen, methane, argon, or combinations thereof. In embodiments of the invention, synthesis gas feed stream 30 includes 5 to 25 mol. % of the inert gas. Synthesis gas feed stream 30 may comprise natural gas derived from a natural gas well, a shale gas well, gasification of biomass and/or coal, or combinations thereof. A flowrate of first synthesis gas stream 28 may be no more than 75% of synthesis gas feed stream 30. A flowrate of second synthesis gas stream 16 may be no more than 25% of synthesis gas feed stream 30. In embodiments of the invention, a flowrate ratio of first synthesis gas stream 28 to second synthesis gas stream 16 can be directly proportional to the reactor volumetric ratio of primary methanol synthesis unit 101 to secondary methanol synthesis unit 105. According to embodiments of the invention, the flowrate ratio of first synthesis gas stream 28 to second synthesis gas stream 16 is from 3:1 to 4:1.

According to embodiments of the invention, as shown in block 202, method 200 includes, in primary methanol synthesis unit 101, subjecting first synthesis gas stream 28 to reaction conditions sufficient to produce first effluent stream 14. In embodiments of the invention, the reaction conditions at block 202 includes a reaction temperature of 200 to 300° C. and all ranges and values there between including ranges of 200 to 205° C., 205 to 210° C., 210 to 215° C., 215 to 220° C., 220 to 225° C., 225 to 230° C., 230 to 235° C., 235 to 240° C., 240 to 245° C., 245 to 250° C., 250 to 255° C., 255 to 260° C., 260 to 265° C., 265 to 270° C., 270 to 275° C., 275 to 280° C., 280 to 285° C., 285 to 290° C., 290 to 295° C., and 295 to 300° C. The reaction conditions at block 202 may further include a reaction pressure of 70 to 120 bar and all ranges and values there between including ranges of 70 to 75 bar, 75 to 80 bar, 80 to 85 bar, 85 to 90 bar, 90 to 95 bar, 95 to 100 bar, 100 to 105 bar, 105 to 110 bar, 110 to 115 bar, and 115 to 120 bar. The reaction conditions at block 202 may further include a space velocity in a range of 4000 to 45000 hr⁻¹ and all ranges and values there between including ranges 4000 to 6000 hr⁻¹, 6000 to 8000 hr⁻¹, 8000 to 10000 hr⁻¹, 10000 to 12000 hr⁻¹, 12000 to 14000 hr⁻¹, 14000 to 16000 hr⁻¹, 16000 to 18000 hr⁻¹, 18000 to 20000 hr⁻¹, 20000 to 22000 hr⁻¹, 22000 to 24000 hr⁻¹, 24000 to 26000 hr⁻¹, 26000 to 28000 hr⁻¹, 28000 to 30000 hr⁻¹, 30000 to 32000 hr⁻¹, 32000 to 34000 hr⁻¹, 34000 to 36000 hr⁻¹, 36000 to 38000 hr⁻¹, 38000 to 40000 hr⁻¹, 40000 to 42000 hr⁻¹, 42000 to 44000 hr⁻¹, and 44000 to 45000 hr⁻¹. In embodiments of the invention, first effluent stream 14 comprises methanol, hydrogen, carbon monoxide, carbon dioxide, the inert gas, or combinations thereof. First effluent stream 14 may include 2 to 20 mol. % methanol and all ranges and values there between including ranges of 2 to 4 mol. %, 4 to 6 mol. %, 6 to 8 mol. %, 8 to 10 mol. %, 10 to 12 mol. %, 12 to 14 mol. %, 14 to 16 mol. %, 16 to 18 mol. %, and 18 to 20 mol. %.

According to embodiments of the invention, as shown in block 203, method 200 includes, in secondary methanol synthesis unit 105, subjecting second synthesis gas stream 16 to reaction conditions sufficient to produce second effluent stream 19. In embodiments of the invention, the reaction conditions at block 203 can be the same or different from the reaction conditions at block 202. The reaction conditions at block 203 may include a reaction temperature of 200 to 300° C. and all ranges and values there between including ranges of 200 to 205° C., 205 to 210° C., 210 to 215° C., 215 to 220° C., 220 to 225° C., 225 to 230° C., 230 to 235° C., 235 to 240° C., 240 to 245° C., 245 to 250° C., 250 to 255° C., 255 to 260° C., 260 to 265° C., 265 to 270° C., 270 to 275° C., 275 to 280° C., 280 to 285° C., 285 to 290° C., 290 to 295° C., and 295 to 300° C. The reaction conditions at block 203 may include a reaction pressure of 70 to 120 bar and all ranges and values there between including ranges of 70 to 75 bar, 75 to 80 bar, 80 to 85 bar, 85 to 90 bar, 90 to 95 bar, 95 to 100 bar, 100 to 105 bar, 105 to 110 bar, 110 to 115 bar, and 115 to 120 bar. The reaction conditions at block 203 may include a space velocity in a range of 4000 to 45000 hr⁻¹ and all ranges and values there between including ranges 4000 to 6000 hr⁻¹, 6000 to 8000 hr⁻¹, 8000 to 10000 hr⁻¹, 10000 to 12000 hr⁻¹, 12000 to 14000 hr⁻¹, 14000 to 16000 hr⁻¹, 16000 to 18000 hr⁻¹, 18000 to 20000 hr⁻¹, 20000 to 22000 hr⁻¹, 22000 to 24000 hr⁻¹, 24000 to 26000 hr⁻¹, 26000 to 28000 hr⁻¹, 28000 to 30000 hr⁻¹, 30000 to 32000 hr⁻¹, 32000 to 34000 hr⁻¹, 34000 to 36000 hr⁻¹, 36000 to 38000 hr⁻¹, 38000 to 40000 hr⁻¹, 40000 to 42000 hr⁻¹, 42000 to 44000 hr⁻¹, and 44000 to 45000 hr⁻¹. In embodiments of the invention, second effluent stream 19 comprises 2 to 20 mol. % methanol. Second effluent stream 19 may further include unreacted hydrogen, unreacted carbon oxides, the inert gas, water, side products, or combinations thereof.

According to embodiments of the invention, as shown in block 204, method 200 further includes flowing first effluent stream 14 and/or second effluent stream 19 to first separation unit 109. In embodiments of the invention, as shown in block 205, method 200 further includes separating, in first separation unit 109, first effluent stream 14 and/or second effluent stream 19 to form (i) first unreacted syngas stream 23 comprising unreacted carbon oxides (including carbon monoxide and carbon dioxide), unreacted hydrogen, and the inert gas, and (ii) first crude methanol stream 22 comprising primarily methanol. In embodiments of the invention, first unreacted syngas stream 23 includes 5 to 35 mol. % of the inert gas and all ranges and values there between including ranges of 5 to 8 mol. %, 8 to 11 mol. %, 11 to 14 mol. %, 14 to 17 mol. %, 17 to 20 mol. %, 20 to 23 mol. %, 23 to 26 mol. %, 26 to 29 mol. %, 29 to 32 mol. %, and 32 to 35 mol. %. First crude methanol stream 22 may include 50 to 85 mol. % methanol and all ranges and values there between including ranges of 50 to 55 mol. %, 55 to 60 mol. %, 60 to 65 mol. %, 65 to 70 mol. %, 70 to 75 mol. %, 75 to 80 mol. %, and 80 to 85 mol. %.

According to embodiments of the invention, as shown in block 206, method 200 further includes dividing first unreacted syngas stream 23 into first recycle stream 24 and purge gas separation unit (PGSU) first feed gas stream 25. In embodiments of the invention, a flow rate ratio of purge gas separation unit (PGSU) first feed gas stream 25 to first recycle stream 24 is in a range of 0 to 20% and all ranges and values there between including ranges of 0 to 2%, 2 to 4%, 4 to 6%, 6 to 8%, 8 to 10%, 10 to 12%, 12 to 14%, 14 to 16%, 16 to 18%, and 18 to 20%. In embodiments of the invention, as shown in block 207, method 200 further includes separating purge gas separation unit (PGSU) first feed gas stream 25 in membrane separation unit 110 to form (i) permeate gas stream 27 comprising primarily carbon oxides (including carbon dioxide and carbon monoxide), and hydrogen and (ii) residue gas stream 26 comprising primarily the inert gas. Permeate gas stream 27 may include 80 to 99 mol. % carbon oxides and hydrogen, collectively.

According to embodiments of the invention, as shown in block 208, method 200 further includes combining permeate gas stream 27 with crude synthesis gas stream 50 to form synthesis gas feed stream 30. Synthesis gas feed stream 30 may be divided to form first synthesis gas stream 28 and second synthesis gas stream 16. In embodiments of the invention, first recycle stream 24 is combined with first synthesis gas stream 28 to form first feed stream 11 prior to being flowed into primary methanol synthesis unit 101. First feed stream 11 may be further preheated by first preheater 103 prior to being flowed into primary methanol synthesis unit 101.

As an alternative or in addition to flowing second effluent stream 19 to first separation unit 109 at block 204, as shown in block 209, method 200 may include separating second effluent stream 19 in second separation unit 120 to form second crude methanol stream 31 comprising primarily methanol and second unreacted syngas stream 32 comprising unreacted carbon oxides, unreacted hydrogen, the inert gas, or combinations thereof. In embodiments of the invention, second crude methanol stream 31 comprises 50 to 85 mol. % methanol and all ranges and values there between including ranges of 50 to 55 mol. %, 55 to 60 mol. %, 60 to 65 mol. %, 65 to 70 mol. %, 70 to 75 mol. %, 75 to 80 mol. %, and 80 to 85 mol. %. Second unreacted syngas stream 32 may include 2 to 25 mol. % of the inert gas and all ranges and values there between including ranges of 2 to 5 mol. %, 5 to 8 mol. %, 8 to 11 mol. %, 11 to 14 mol. %, 14 to 17 mol. %, 17 to 20 mol. %, 20 to 23 mol. %, and 23 to 25 mol. %.

In embodiments of the invention, as shown in block 210, method 200 further includes dividing second unreacted syngas stream to form second recycle stream 33 and purge gas separation unit (PGSU) second feed gas stream 34. According to embodiments of the invention, as shown in block 211, method 200 further includes separating both purge gas separation unit (PGSU) first feed gas stream 25 and purge gas separation unit (PGSU) second feed gas stream 34 in membrane separation unit 110 to produce (I) permeate gas stream 27 comprising primarily carbon oxides and hydrogen, collectively, and (II) residue gas stream 26 comprising primarily hydrogen and the inert gas. In embodiments of the invention, as shown in block 212, method 200 further still includes combining permeate gas stream 27 with crude synthesis gas stream 50 to form synthesis gas feed stream 30. In embodiments of the invention, second recycle stream 33 is combined with second synthesis gas stream 16 to form second feed stream 17. Second feed stream 17 may be flowed to secondary methanol synthesis unit 105. Second recycle stream 33 may be compressed by second recycle compressor 121 before it is combined with second synthesis gas stream 16.

Although embodiments of the present invention have been described with reference to blocks of FIG. 3, it should be appreciated that operation of the present invention is not limited to the particular blocks and/or the particular order of the blocks illustrated in FIG. 3. Accordingly, embodiments of the invention may provide functionality as described herein using various blocks in a sequence different than that of FIG. 3.

In the context of the present invention, at least the flowing 15 embodiments are described. Embodiment 1 is a method of producing methanol. The method includes providing a synthesis gas feed stream comprising carbon oxides, hydrogen, and an inert gas. The method includes dividing the synthesis gas feed stream to form a first synthesis gas stream and a second synthesis gas stream. The method includes in a primary methanol synthesis unit, subjecting the first synthesis gas stream to reaction conditions sufficient to produce a first effluent stream comprising methanol, unreacted carbon oxides, unreacted hydrogen, and a first portion of the inert gas. The method includes in a secondary methanol synthesis unit, subjecting the second synthesis gas stream to reaction conditions sufficient to produce a second effluent stream comprising methanol, unreacted carbon oxides, unreacted hydrogen, and a second portion of the inert gas. The method further includes separating the methanol and/or at least some inert gas from the first effluent stream and/or the second effluent stream to produce a first recycle stream comprising primarily unreacted carbon oxides, unreacted hydrogen and the inert gas collectively, and a permeate gas stream comprising primarily unreacted carbon oxides and unreacted hydrogen, collectively. The method further still includes flowing the first recycle stream and permeate gas stream to the primary methanol synthesis unit. Embodiment 2 is the method of embodiment 1, wherein the permeate gas stream is flowed back to mix with syngas stream feeding both the primary methanol synthesis unit and the secondary methanol synthesis unit. Embodiment 3 is the method of any of embodiments 1 and 2, wherein the first recycle stream comprises 5 to 35 mol. % of inert gas. Embodiment 4 is the method of any of embodiments 1 to 3, wherein the separating step includes separating the first effluent stream and the second effluent stream in a first separation unit to produce a first unreacted syngas stream and a first crude methanol stream; dividing the first unreacted syngas stream to form the first recycle stream and a purge gas separation unit (PGSU) first feed gas stream; and separating the purge gas separation unit (PGSU) first feed gas stream in an inert separation unit to form (i) the permeate gas stream comprising primarily carbon oxides and hydrogen, collectively, and (ii) a residue gas stream comprising primarily the inert gas. Embodiment 5 is the method of embodiment 1, wherein the separating step includes separating the first effluent stream in a first separation unit to produce a first unreacted syngas stream and a first crude methanol stream; separating the second effluent stream in a second separation unit to produce a second unreacted syngas stream and a second crude methanol stream; dividing the first unreacted syngas stream to form the first recycle stream and a purge gas separation unit (PGSU) first feed gas stream; dividing the second unreacted syngas stream to form the second recycle stream and a purge gas separation unit (PGSU) second feed gas stream; and separating the purge gas separation unit (PGSU) first feed gas stream and the purge gas separation unit (PGSU) second feed gas stream in an inert separation unit to form (i) permeate gas stream comprising primarily carbon oxides and hydrogen, collectively and (ii) a residue gas stream comprising primarily the inert gas. Embodiment 6 is the method of embodiment 5, wherein the second recycle stream is flowed back to the secondary methanol synthesis unit. Embodiment 7 is the method of any of embodiments 1 to 6, wherein the first synthesis gas stream comprises greater than or equal to 75% of the synthesis gas feed stream and the second synthesis gas stream comprises less than or equal to 25% of the synthesis gas feed stream. Embodiment 8 is the method of any of embodiments 1 to 7, wherein the primary methanol synthesis unit comprises a catalyst comprising Cu, Zn, Al₂O₃, or combinations thereof. Embodiment 9 is the method of embodiment 8, wherein the secondary methanol synthesis unit includes the same or substantially the same catalyst as the catalyst of primary methanol synthesis unit. Embodiment 10 is the method of any of embodiments 1 to 9, wherein the secondary methanol synthesis unit has a reactor volume less than or equal to 25% of a reactor volume of the primary methanol synthesis unit. Embodiment 11 is the method of any of embodiments 1 to 10, wherein the inert gas is selected from the group consisting of nitrogen, argon, methane, and combinations thereof. Embodiment 12 is the method of any of embodiments 1 to 11, wherein the reaction conditions in the primary methanol synthesis unit and/or the secondary methanol synthesis unit include a reaction temperature of 200 to 300° C., a reaction pressure of 70 to 120 bar, and a space velocity in a range of 4000 to 45000 hr⁻¹. Embodiment 13 is the method of any of embodiments 1 to 12, wherein the reaction conditions in the primary methanol synthesis unit are the same or substantially the same as the reaction conditions in the secondary methanol synthesis unit. Embodiment 14 is the method of any of embodiments 1 to 13, wherein the secondary methanol synthesis unit comprises one or more adiabatic or isothermal reactors in series. Embodiment 15 is the method of any of embodiments 1 to 14, wherein the synthesis gas feed stream is derived from a natural gas well, a shale gas well, gasification of biomass and/or coal, or combinations thereof.

The systems and processes described herein can also include various equipment that is not shown and is known to one of skill in the art of chemical processing. For example, some controllers, piping, computers, valves, pumps, heaters, thermocouples, pressure indicators, mixers, heat exchangers, and the like may not be shown.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A method of producing methanol, the method comprising:
providing a synthesis gas feed stream comprising carbon oxides, hydrogen, and an inert gas;
dividing the synthesis gas feed stream to form a first synthesis gas stream and a second synthesis gas stream;
in a primary methanol synthesis unit, subjecting the first synthesis gas stream to reaction conditions sufficient to produce a first effluent stream comprising methanol, unreacted carbon oxides, unreacted hydrogen, and a first portion of the inert gas;
in a secondary methanol synthesis unit, subjecting the second synthesis gas stream to reaction conditions sufficient to produce a second effluent stream comprising methanol, unreacted carbon oxides, unreacted hydrogen, and a second portion of the inert gas;
separating the methanol and/or at least some inert gas from the first effluent stream and/or the second effluent stream to produce a first recycle stream comprising primarily unreacted carbon oxides, unreacted hydrogen and the inert gas collectively, and a permeate gas stream comprising primarily unreacted carbon oxides and unreacted hydrogen, collectively; and
flowing the first recycle stream and permeate gas stream to the primary methanol synthesis unit.

2. The method of claim 1, wherein the permeate gas stream is flowed back to mix with syngas stream feeding both the primary methanol synthesis unit and the secondary methanol synthesis unit.

3. The method of claim 1, wherein the first recycle stream comprises 5 to 35 mol. % of inert gas.

4. The method of claim 1, wherein the separating step comprises:
separating the first effluent stream and the second effluent stream in a first separation unit to produce a first unreacted syngas stream and a first crude methanol stream;
dividing the first unreacted syngas stream to form the first recycle stream and a purge gas separation unit (PGSU) first feed gas stream; and
separating the purge gas separation unit (PGSU) first feed gas stream in an inert separation unit to form (i) the permeate gas stream comprising primarily carbon oxides and hydrogen, collectively, and (ii) a residue gas stream comprising primarily the inert gas.

5. The method of claim 1, wherein the separating step comprises:
separating the first effluent stream in a first separation unit to produce a first unreacted syngas stream and a first crude methanol stream;
separating the second effluent stream in a second separation unit to produce a second unreacted syngas stream and a second crude methanol stream;
dividing the first unreacted syngas stream to form the first recycle stream and a purge gas separation unit (PGSU) first feed gas stream;
dividing the second unreacted syngas stream to form the second recycle stream and a purge gas separation unit (PGSU) second feed gas stream;
separating the purge gas separation unit (PGSU) first feed gas stream and the purge gas separation unit (PGSU) second feed gas stream in an inert separation unit to form (i) permeate gas stream comprising primarily carbon oxides and hydrogen, collectively and (ii) a residue gas stream comprising primarily the inert gas.

6. The method of claim 5, wherein the second recycle stream is flowed back to the secondary methanol synthesis unit.

7. The method of claim 1, wherein the first synthesis gas stream comprises greater than or equal to 75% of the synthesis gas feed stream and the second synthesis gas stream comprises less than or equal to 25% of the synthesis gas feed stream.

8. The method of claim 1, wherein the primary methanol synthesis unit comprises a catalyst comprising Cu, Zn, Al₂O₃, or combinations thereof.

9. The method of claim 8, wherein the secondary methanol synthesis unit includes the same or substantially the same catalyst as the catalyst of primary methanol synthesis unit.

10. The method of claim 1, wherein the secondary methanol synthesis unit has a reactor volume less than or equal to 25% of a reactor volume of the primary methanol synthesis unit.

11. The method of claim 1, wherein the inert gas is selected from the group consisting of nitrogen, argon, methane, and combinations thereof.

12. The method of claim 1, wherein the reaction conditions in the primary methanol synthesis unit and/or the secondary methanol synthesis unit include a reaction temperature of 200 to 300° C., a reaction pressure of 70 to 120 bar, and a space velocity in a range of 4000 to 45000 hr⁻¹.

13. The method of claim 1, wherein the reaction conditions in the primary methanol synthesis unit are the same or substantially the same as the reaction conditions in the secondary methanol synthesis unit.

14. The method of claim 1, wherein the secondary methanol synthesis unit comprises one or more adiabatic or isothermal reactors in series.

15. The method of claim 1, wherein the synthesis gas feed stream is derived from a natural gas well, a shale gas well, gasification of biomass and/or coal, or combinations thereof.

16. The method of claim 2, wherein the inert gas is selected from the group consisting of nitrogen, argon, methane, and combinations thereof.

17. The method of claim 2, wherein the reaction conditions in the primary methanol synthesis unit and/or the secondary methanol synthesis unit include a reaction temperature of 200 to 300° C., a reaction pressure of 70 to 120 bar, and a space velocity in a range of 4000 to 45000 hr⁻¹.

18. The method of claim 2, wherein the reaction conditions in the primary methanol synthesis unit are the same or substantially the same as the reaction conditions in the secondary methanol synthesis unit.

19. The method of claim 2, wherein the secondary methanol synthesis unit comprises one or more adiabatic or isothermal reactors in series.

20. The method of claim 2, wherein the synthesis gas feed stream is derived from a natural gas well, a shale gas well, gasification of biomass and/or coal, or combinations thereof.
